(19) United States
(12) Patent Application Publication
(10) Pub. No.: US 2019/0125483 A1
(43) Pub. Date: May 2, 2019

(54) SURGICAL MARKER

(71) Applicant: CLÍNICA DE ORTODONCIA Y CIRUGÍA MAXILOFACIAL, S.L., Santa Cruz de Tenerife (ES)

(72) Inventor: Carlos MARTÍNEZ GIMENO, Santa Cruz de Tenerife (ES)

(21) Appl. No.: 16/091,648

(22) PCT Filed: Mar. 23, 2017
(86) PCT No.: PCT/ES2017/070171

§ 371 Date: Oct. 5, 2018

(30) Foreign Application Priority Data
Apr. 6, 2016 (EP) 16382149.9

(51) Int. Cl. A61B 90/00; A61M 37/00; B43K 23/08; B43K 27/02; B43K 29/08; B43K 29/00

(52) U.S. Cl. CPC A61B 90/39; A61M 37/0084; B43K 23/08; B43K 27/02; A61B 2017/00473; B43K 29/00; A61B 2090/3933; A61B 2090/395; A61B 2090/3987; B43K 29/08

(57) ABSTRACT

The invention relates to a surgical marker (1) comprising an elongate handle (2) that can be gripped with the hand in the manner of a pen, and means provided on the handle (2) for interchangeably attaching interchangeable points (3, 4, 5), the interchangeable points comprising: a point made of a porous material that can be soaked in ink (3), for marking on skin; a graphite point (4) for marking on bone; and an injector point (5) for precision-marking points to outline skin grafts by means of an injection into the dermis that is not erased with surgical washing.

### OBJECT OF THE INVENTION

The present invention relates to a surgical marker. The corresponding field is that of medical-surgical instruments.

### BACKGROUND OF THE INVENTION

Current surgical markers basically consist of sterile pens with different tip thickness and colours, using different ink types (methylene blue, gentian violet, etc.) which mainly work on dry surfaces and which fade or disappear with surgical washing. None of them can paint or mark moist surfaces or bone in a lasting manner.

In order to mark beneath the epidermis markers are known such as that described in U.S. Pat. No. 4,789,582, which comprises a needle cartridge for a surgical pen or tattooing tool with a reservoir filled with a liquid pigment adapted to be discharged to the skin through a needle with three simultaneously acting tips. However, it requires a specific air-driven pen.

Document U.S. Pat. No. 5,496,304 discloses a device and method comprising a member for penetrating the outermost layer of the epidermis and a reservoir supplying a marking agent, such that as the penetrating member enters the epidermis the marking agent is introduced under the outermost layer thereof, using as a marker, for example, methylene blue. The penetrating member s shaped as a wheel with tips to mark lines. It is therefore not suitable for marking points or marks that do not require lengthy displacements of the marker, such as in anatomical parts with folds or reduced space, and can only be used on the skin. A similar construction and advantages and drawbacks are present in the marker of U.S. Pat. No. 5,713,890 and that of US2004/0116907.

Document U.S. Pat. No. 5,909,978 describes a marker comprising a cartridge with inks, airtight except at an opening in the distal end—to receive the tip of a marker—and a ventilation orifice, wherein the walls of the pen included in the marker are flexible in order to eject the ink, which is also regulated by partially covering the ventilation orifice. The purpose of this is to force the exit and generate a constant flow of ink, preventing the drawback of tip obstruction or failure of operation because the tip is mixed with grease or tissue remains; however, this marker cannot inject subcutaneously.

Also known is U.S. Pat. No. 6,197,034, which describes a marker that can be used on soft and hard mammalian tissue, as well as a pen containing a medical marking device and methods for using said pens to make lines and marks on soft and hard mammalian tissue. The pen can be used for other surgical marking applications and is used as a normal writing implement. It comprises a housing with an inner reservoir containing inks and two ends, one of which is closed while the opposite end has an extended member with a groove, and a hollow tube coaxial with the groove. Inside the reservoir is a vertically movable mass joined to a stem with a length somewhat greater than that of the hollow tube, such that the mass blocks the opening of the inks unless the stem is pushed from above. This is identical to certain old linear drawing devices and has the same drawbacks of the ink drying in the hollow tube, as well as uncontrolled ink outlet in certain situations.

Document US2013/0197358 describes another surgical marker comprising: a pen body with a first chamber containing a powder colouring, a second chamber separated from the first chamber containing a solvent, where the first and second chamber are selectively placed with fluid communication with each other, and a tip in fluid communication with the first or second chamber, wherein at least one powder colouring and the solvent, when mixed, form a colouring solution that can be discarded on the surface of the skin or tissue and can be viewed simultaneously by a user without assistance and with a fluorescent image generator. It can be used to mark areas which previous required fluorescent light generators to be seen, but maintains the specific construction with the various chambers, and does not ensure permanence of the marking with surgical washing.

Also known is document US2015/0119866, which describes a surgical marker for laparoscopic interventions that is connected to a rod and can be pushed through a laparoscopy cannula to the target tissue to mark. The marker can adjust (reduce) its length by moving the location of the ink reservoir from its position in a typical marker to a location on a cover, and comprises a connector with a cap formed therein to receive the pin of a rod and a tip to contain and distribute the ink on the tissue.

Document U.S. Pat. No. 5,665,092 describes a surgical marker for bones comprising a tubular guide cannula, a tube that can be inserted in the cannula with a proximal end and a distal end, a flexible and movable rod that extends along and inside the tube, and a marking tip connected to a proximal end of the flexible rod and extending beyond the proximal end of the tube, which allows marking the operation site accurately with minimal pain to the patient. Marking is produced by bone erosion and the marker overall has a complex construction and handling.

All of these surgical markers have a specific use, either for subcutaneous marking, linear or otherwise, bone erosion or simple marking, such that their versatility is very limited. In addition, the permanence of the marking with surgical washing is ensured in only a few of them.

### DESCRIPTION OF THE INVENTION

The surgical marker of the invention has a configuration with small multi-surface tips that are exchangeable, allowing to use only those needed according to the technique and tissue to mark, and allowing surgical field markings that are not possible with current devices.

In addition, the marking performed in each case is resistant to surgical washing and provides a lasting marking.

According to the invention, the marker comprises:
an elongated handle that can be held with the hand as a normal marker or writing implement; and
exchangeable attaching means for exchangeable tips, provided on said handle and at the tips;

Where the exchangeable tips comprise:
a tip of a porous material that can be soaked in ink, similar to that of a marker, for marking skin;
a graphite tip for marking bone, even if the same is wet or has blood, which allows using saws with irrigation without fading and having to reapply the marking; and
an injection tip for precision markings of subcutaneous points in the design of skin plasties by injection in the dermis, which is resistant to surgical washing, such that several tips can be used in a single surgical intervention according to the technique employed.

In addition, the marking performed in each case lasts during the work, as the tip of porous material soaked in ink is used with moisture-resistant ink, the graphite tip allows marking on bone even when wet and allows using saws with irrigation without fading and requiring remarking, and in subcutaneous injection the skin itself protects the ink.

### BRIEF DESCRIPTION OF THE DRAWINGS

FIG. 1 shows a side view of the marker of the invention with the various tips thereof, where the covers of said tips are sectioned for purposes of clarity.

FIGS. 2, 3 and 4 respectively show a side view of the marker of the invention with the tip of porous material soakable in ink, the graphite tip and the injection tip mounted, marking the concealed lines and with the covers of the tips sectioned.

FIG. 5 shows a side, elevation and bottom plan view of the marker of the invention with the injection tip mounted, showing by concealed lines in the elevation view the male protrusion of the exchangeable tip attachment means to the handle inserted in the female notch.

FIG. 6 shows an enlarged view of the clipping of one of the tips to the handle of the marker of the invention. This clipping is the same for all the tips.

### DESCRIPTION OF AN EMBODIMENT

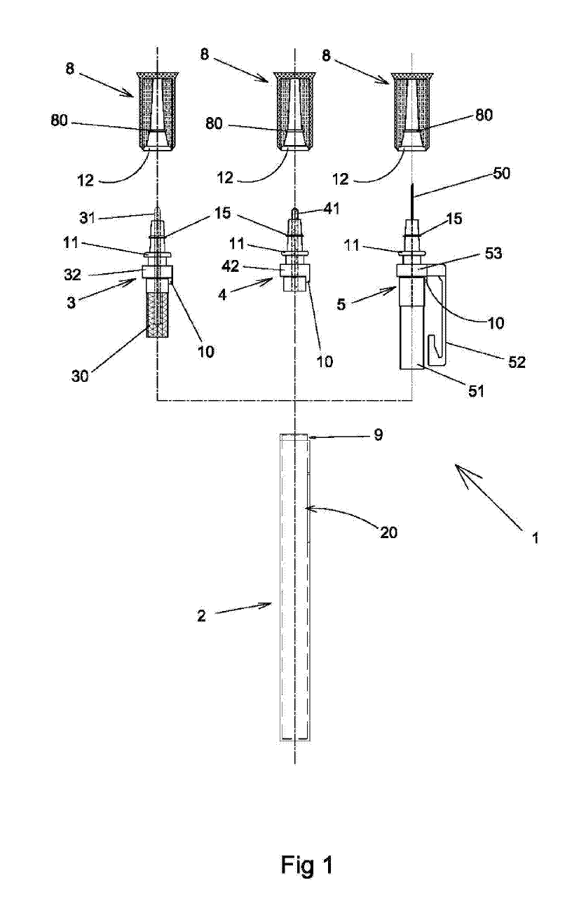

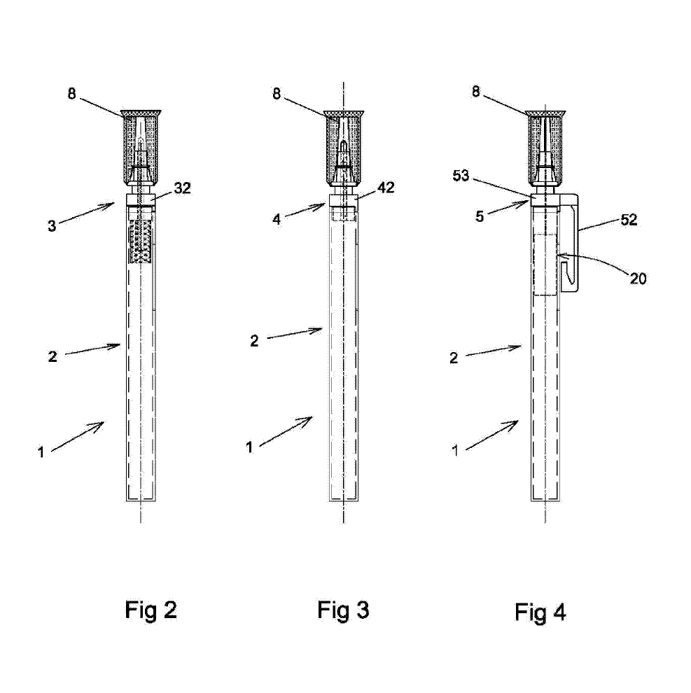

The surgical marker (1) of the invention comprises (see FIG. 1):
a handle (2) in the form of a marker body, tubular, that can be held in the hand, and
exchangeable attachment means for exchangeable tips (3, 4, 5) arranged on said handle (2) and at the tips (3, 4, 5),
   where the exchangeable tips comprise:
a tip (3) made of a porous material soakable in ink for marking skin,
a graphite tip (4) for marking bone, even if it is wet or has blood,
an injection tip (5) for precision marking of points to design skin plasties by injection in the dermis that do not fade with surgical washing. This tip is particularly useful to mark skin flaps with precise geometry, such as in cleft lips, other plasties with precise geometry and to perform otoplasties.

All of the above such that in a single surgical intervention several tips can be used depending on the technique to perform.

The tips (3) made of a porous material soakable in ink comprise an applicator (31) for said material and a reservoir (30) with the same or similar material, increasing the amount of ink contained and therefore the duration thereof. The applicator (31) and the reservoir (30) are mounted on a first support (32) where the exchangeable attachment means to the handle (2) are implemented. In addition, said tips (3) of a porous material soakable in ink are soaked in moisture-resistant ink and comprise inks of several colours and/or thickness, including at least three different thicknesses: fine, medium and thick.

The graphite tip (4) instead comprises a graphite portion (41) with at least HD hardness, such as to allow an effective bone marking, and is mounted on a second support (42) where the exchangeable attachment means to the handle (2) are implemented.

With regard to the injection tip (5), ideally it comprises a hollow needle (50) for subcutaneous injection of the ink, in communication with an elastic reservoir (51) that can be compressed through an orifice (20) provided on the handle (2) that allows pressing said elastic reservoir (51) applying sufficient pressure to inject the ink subcutaneously through the hollow needle (51). Said hollow needle (50) and the reservoir (51) are mounted on a third support (53) where the exchangeable attachment means to the handle (2) are implemented. In addition, said third support (53) preferably comprises a flexible elastic lever (52) that can recover its position coinciding with the orifice (20) and with the reservoir (51) in order to compress the latter more easily, the orifice (20) located on the end of the handle (2) where the exchangeable attachment means of the tips (3, 4, 5) are provided, and at the same position as the reservoir (51), such that a reservoir with sufficient volume is obtained, yet not too big for the ink to dry or the pressure exerted to be dispersed. The ink used in the injection point (5) can be methylene blue or gentian violet, which will not fade with surgical washing, remaining at the initially intended position even if the wound is already open.

The exchangeable attachment means of the exchangeable tips (3, 4, 5) provided on the handle (2) comprise in this preferred embodiment (see FIG. 6) a removable annular clip (19) as well as a female notch (9) and a complementary male protrusion (10), such that the union is not rotary and connection is simple and effective Said female notch (9) is provided on the handle (2) and the male protrusion (10) is provided on the tips (3, 4, 5) as shown in the figures, or vice versa.

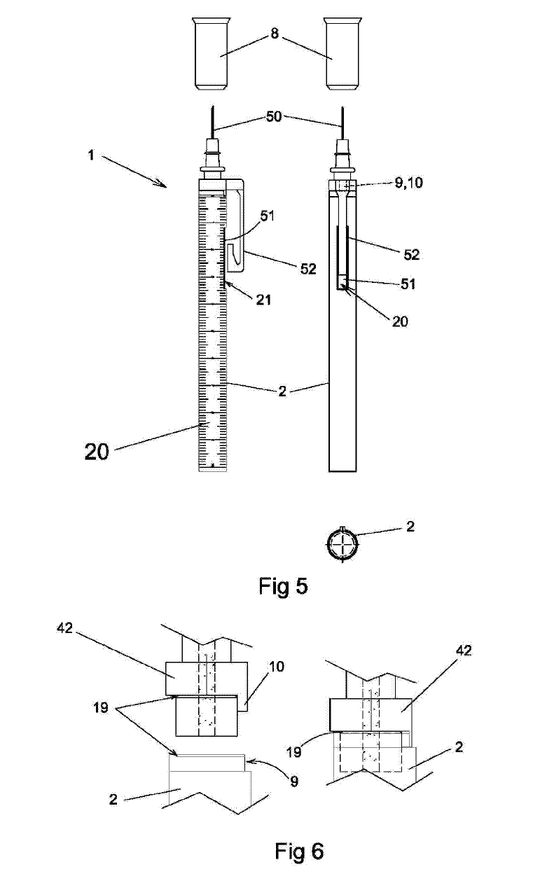

In addition the handle (2) comprises a ruler (21) (see FIG. 5) to measure the markings, which can be used with the tips (3, 4, 5) mounted or not mounted on the marker (1). Said ruler (21) ideally has millimetre gradings and can be implemented in the handle (2) by printing and/or moulding.

The tips (3, 4, 5) ideally comprise a lid (8) to prevent the porous material from drying and/or to protect the graphite and the injection needle. Said lid (8) is attached by fitting means to the corresponding tip (3, 4, 5), the fitting means thereof have a smaller tensile resistance than the exchangeable attachment means of the tips (3, 4, 5) (than the annular clip (19) in this example), in order to take out the lid (8) without removing the tip mounted on the handle (2). Said fitting means comprise for example an annular protrusion (11) and an annular fitting (12) for said annular protrusion (11), said annular protrusion (11) being disposed on the tip (3, 4, 5) and the annular fitting in the lid (8), or vice versa.

In addition, the tip (3, 4, 5) comprises a frustoconical sealing lip (15) arranged against an inner recess (80) of the lid (8) to prevent air from entering and drying the ink.

Lastly, the handle (2) and/or the tips (3, 4, 5) are made of plastic to reduce costs, and the tips (3, 4, 5) comprise a preliminary sterilisation treatment and a sterile protection packaging, not shown, for immediate use.

Having sufficiently described the nature of the invention and the embodiment thereof, it should be noted that the arrangements described above and shown in the accompanying drawings are subject to changes in the details thereof provided the fundamental design is not affected.

1. Surgical marker (1) characterised in that it comprises:
an elongated handle (2) for holding with the hand as a writing instrument, and
exchangeable attachment means for exchangeable tips (3, 4, 5), provided on said handle and on the tips (3, 4, 5),
where the exchangeable tips comprise:
a tip (3) made of a porous material soakable in ink for marking skin,
a graphite tip (4) for marking bone, and
an injection tip (5) for precision marking of points to design skin plasties by injection in the dermis.

2. Surgical marker (1) according to claim 1 characterised in that the tips (3) made of a porous material soakable in ink comprise an applicator (31) for said material and a reservoir (30); the applicator (31) and the reservoir (30) mounted on a first support (32) in which are implemented the exchangeable attachment means to the handle (2).

3. Surgical marker (1) according to claim 1 characterised in that the tips (3) made of a porous material soakable in ink are soaked in moisture-resistant ink.

4. Surgical marker (1) according to claim 1 characterised in that it comprises tips (3) made of a porous material soakable in ink with different thicknesses.

5. Surgical marker (1) according to claim 1 characterised in that the graphite tip (4) comprises a portion of graphite (41) with at least HD hardness, mounted on a second support (42) in which are implemented the exchangeable attachment means to the handle (2).

6. Surgical marker (1) according to claim 1 characterised in that the injection tip (5) comprises a hollow needle (50) for subcutaneous injection of the ink in communication with an elastic reservoir (51) that can be compressed through an orifice (20) provided on the handle (2); the hollow needle (50) and the reservoir (51) mounted on a third support (53) in which are implemented the exchangeable attachment means to the handle (2).

7. Surgical marker (1) according to claim 6 characterised in that the third support (53) comprises a flexible elastic lever (52) that can restore its position in coincidence with the orifice (20) to compress the reservoir.

8. Surgical marker (1) according to claim 6 characterised in that the ink used in the injection point (5) is selected among:
methylene blue, or
gentian violet.

9. Surgical marker (1) according to claim 1 characterised in that the exchangeable attachment means provided on the handle (2) comprise a removable annular clip (19) and a female notch (9) and a complementary anti-rotation male protrusion (10); where the female notch (9) is provided on the handle (2) and the male protrusion (10) on the tips (3, 4, 5) or vice versa.

10. Surgical marker (1) according to claim 1 characterised in that the handle (2) comprises a ruler (21) for measuring the markings.

11. Surgical marker (1) according to claim 1 characterised in that the tips (3, 4, 5) comprise a lid (8).

12. Surgical marker (1) according to claim 11 characterised in that the lid (8) is attached by fitting means to the corresponding tip (3, 4, 5), the attachment means of which have a lower tensile resistance than the exchangeable attachment means of the tips (3, 4, 5).

13. Surgical marker (1) according to claim 1 characterised in that the tip (3, 4, 5) comprises a sealing lip (15) placed against an inner recess (80) of the lid (8).

14. Surgical marker (1) according to claim 1 characterised in that the handle (2) and/or the tips (3, 4, 5) are made of plastic materials.

15. Surgical marker (1) according to claim 1 characterised in that the tips (3, 4, 5) comprise a preliminary sterilisation treatment and a sterile protection packaging.
